(19) United States
(12) Patent Application Publication
(10) Pub. No.: US 2005/0240080 A1
(43) Pub. Date: Oct. 27, 2005

(54) Endoscope provided with an fibre optical bundle

(76) Inventors: Ralf Diekmann, Dulmen (DE); Gunther Meinhardt, Meinerzhagen (DE)

Correspondence Address: RANKIN, HILL, PORTER & CLARK LLP, 4080 ERIE STREET, WILLOUGHBY, OH, 44094-7836, US

(21) Appl. No.: 10/527,144

(22) PCT Filed: Aug. 2, 2003
(86) PCT No.: PCT/EP03/08598

§ 371 Date: Mar. 9, 2005

(30) Foreign Application Priority Data
Sep. 12, 2002 (DE) 102 42 607.4

(51) Int. Cl. A61B 1/06

(52) U.S. Cl. 600/182; 600/178; 600/117

(57) ABSTRACT

An endoscope including an image transmitting system and at least one fiber optics bundle which is mounted by its distal end segment in the endoscope and which at its distal end emits light to illuminate the field of view. A proximal end of the bundle is illuminated by a light source. The endoscope also includes, a tapping system for tapping light from the fiber optics bundle and guiding it to a window in the external endoscope wall.

### BACKGROUND OF THE INVENTION

The present invention relates to an endoscope fitted with a fiber optical bundle, hereafter referred to as a fiber optics bundle.

Foremost, such endoscopes are used in endoscopic surgery, in therapy, or diagnosis; they comprise a stem having a distal zone which can be inserted into an opening and a proximal zone which cannot be inserted in such an opening, furthermore, an image transmission system to view the field of observation, and a fiber optics bundle to illuminate the field of observation.

The fiber optics bundle may be coupled to an external light source or be connected to a light source integrated into the endoscope.

Conventionally, the image transmission system consists of an objective lens mounted on the stem end, relay lens elements and an ocular configured in a proximal zone. However, the relay lens elements may be replaced by a fiber optics bundle or an image transducing chip with their pertinent wires. The image transducing chip also may be used in addition to the relay lens elements or the fiber optics bundle, in which case it shall be situated proximally from the elements or bundle. In the latter two cases, a monitor shall replace the ocular.

Surgery using an endoscope of the above type is frequently carried out in darkened rooms in order to provide the surgeon with as much contrast as possible, thus providing, an image, which otherwise is free of incident ambient light, at the ocular or on the monitor.

However, under such low-light level conditions, the attending personnel meet with difficulties in reliably handling both the endoscope and its accessories. Illustratively, it is difficult to hold the endoscope, to plug in/out hoses or actuate flushing cocks and other functional elements when in such a darkened room

The objective of the present invention is to create an endoscope of the above type which can be located and handled more easily and more reliably in a darkened operating room.

### BRIEF SUMMARY OF THE INVENTION

The present invention provides a tapping system tapping light out of a fiber optics bundle and feeding it to a window in the outer endoscope wall, as a result of which, when the light source is ON, light travels from the window to the outside. In this manner, the endoscope is easily located even in darkness, without glare being present, thanks to the narrow dot of light. By using the light from the fiber optics bundle and because, thereby, additional light sources are not needed, problems are avoided regarding bulk and light blocking, as well as shortcomings regarding autoclaving that might arise when using additional light sources. Moreover, this feature circumvents complexity of construction, manufacturing costs and maintenance expenditures that would be entailed when using additional light sources. Again this feature allows central control of the emitted light at the light sources, for instance with respect to brightness or color.

Accordingly, the endoscope external wall is transparent in the region of a coupling where a hookup cable receiving the proximal terminal segment of the fiber optics can be decoupled from the endoscope. In this region, the mutually abutting end faces of the proximal and distal fiber optics segments will not generally seal each other off in a light-tight manner, instead subtending a small gap through which light does escape. This light may pass through the light-passing region in the endoscope outer wall in the vicinity of the coupling and spread into the atmosphere. These features are the simplest embodiment of the principle of the present invention. No further costs beyond configuring one window are incurred to enable light to arrive at the endoscope outside surface.

The tapping system is in the form of optic fiber(s) branched from the fiber optics bundle and running to the window. This configuration again is economical regarding the number of parts used in the implementation of the principle of the present invention because the fiber optics bundle per se already consists of many optical fibers and individual fibers being easily branched off the bundle. Advantageously too, because of their high mechanical compliance, optical fibers may be made to move into endoscope regions that otherwise can be accessed only with difficulty for purposes of illumination. This ability exists only in limited form in other light sources such as LED's; moreover, and as already mentioned above, there might also be light-blocking and autoclaving problems. Again, this design principle allows increasing the number of illuminating sites almost arbitrarily and at low cost.

Additional features of the invention provide that the window shall be configured in the vicinity of the endoscope's functional elements or labelings. Such elements illustratively are shown by a light dot and in this manner are more easily located.

Advantageously, the features of the invention provide that the light issuing through the window illuminate endoscope functional elements or labelings. In this manner, the elements are more easily located and furthermore will be more easily handled or read.

Several tapping systems and windows are provided in the endoscope's external wall and by their configuration subtend a pattern of light dots. Illustratively, the main endoscope axes, the endoscope contour, or the connection cable receiving the proximal end of the light guide can be marked to facilitate rapidly and accurately locating, holding and putting down again of the endoscope or the hookup cable. In a similar manner, various endoscopes or their parts, for instance the grips, may be coded by simple light-dot patterns to allow rapid and reliable recognition and identification.

### BRIEF DESCRIPTION OF THE DRAWING

FIG. 1 illustratively and schematically shows the present invention.

### DETAILED DESCRIPTION OF THE INVENTION

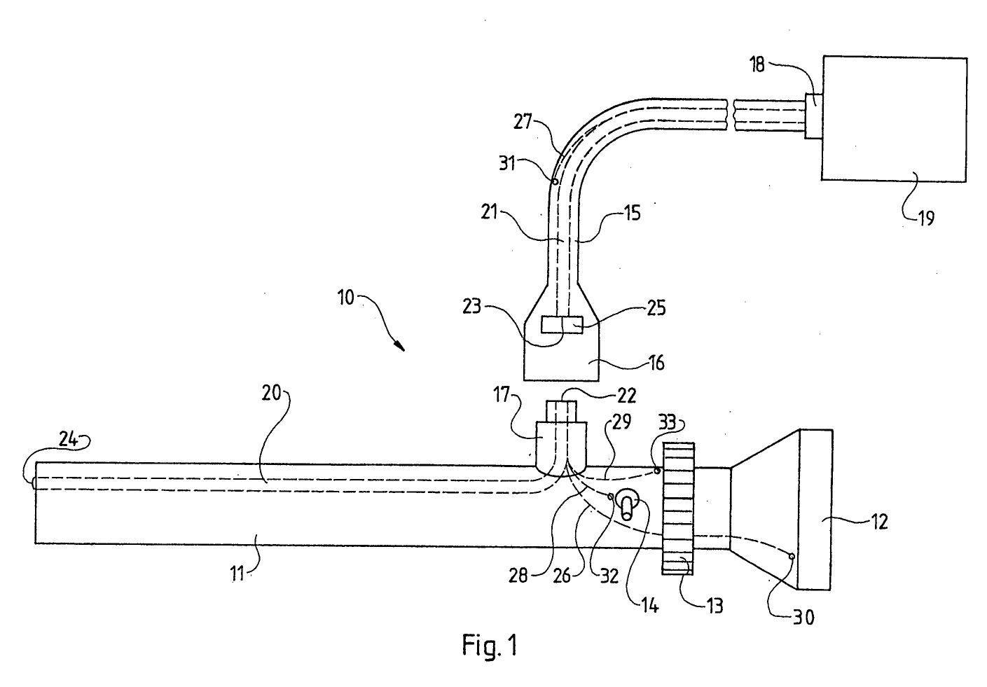

FIG. 1 shows an endoscope 10 of the present invention comprising a stem 11 which may be inserted into a body cavity, further an ocular 12, and an adjustment ring 13 and a switch 14. The endoscope 10 further comprises a fiber optics hookup cable 15 which can be connected by means of a coupling, (i.e. a jack 16) to another coupling, (i.e. a mating plug 17) on the stem 11 and—at its other end—by means of a coupling 18 to a light source 19. In FIG. 1, the fiber optics hookup cable 15 is shown disconnected from the plug 17. The endoscope 10 furthermore comprises a fiber optics bundle having a distal segment 20 and a proximal segment 21. The distal segment 20 of the fiber optics bundle runs in the stem 11 whereas the proximal segment 21 of the fiber optics bundle runs in the fiber optics hookup cable 15.

The proximal segment 21 of the fiber optics bundle guides the light from the light source 19 in the distal direction. The end faces 22 and 23 of the two fiber optics regions are situated narrowly close to each other in the vicinity of the coupling 16, as a result of which light from the proximal segment 21 of the fiber optics bundle passes into its distal segment 20. In the connection state, the end faces 22 and 23 are separated from each other by a narrow gap and in this zone the light does issue from the fiber optics bundle. Light to illuminate the field of view is radiated from the distal end of the fiber optics bundle. Using an image transmission system inside the stem 11 (not shown), the field of view may be observed through the ocular 12.

Within the zone of the coupling 16, the external surface of the endoscope 10 constituted by the coupling sleeve is in the form of an optically transparent window 25. The light issuing from the proximal segment 21 of the fiber optic bundle may be transmitted to the outside from this window.

Individual optic fibers 26, 27, 28 and 29 branch off the optic fiber bundle in the region of the distal segment 20 and the proximal segment 21. The optic fibers run to further windows 30, 31, 32 and 33 which are situated in the external surface of the endoscope and by means of which the light tapped from the optic fiber bundle moves into the surroundings. The windows and their associated optic fibers are configured in such a way that the ocular 12 and the external sheath of the optic fiber hookup cable 15 are marked by light dots, furthermore in that the switch 14 is illuminated and in that the adjustment ring 13 is legible on account of this illumination.

Various embodiment variants are feasible relative to the above discussed embodiment. Illustratively, the distal segment 20 and the proximal segment 21 of the fiber optics may be integrally connected to each other. In that case there shall be no coupling 16.

The fiber optics bundle may be connected to an internal light source instead of an external light source 19, the internal light source illustratively being configured in the endoscope's proximal segment. In this case the coupling 16 again is eliminated.

In addition to, or instead of the elements illuminated through windows in the above discussed embodiment, other functional elements or labelings of the endoscope may be marked or illuminated or read by means of the illumination by appropriately positioning the windows and the associated optic fibers.

Again, several windows and branched optic fibers underneath them may be configured in the external surface in a manner that the radiated light shall form a pattern of light dots on the endoscope external surface, for instance a line representing the endoscope's main axes or its contour. The radiated pattern also may constitute a light dot code by means of which the endoscope or a functional element may be unambiguously identified.

The windows may be designed to be darkened or closed as needed, as a result of which the issuing light flux may be metered. In this manner glare can be substantially eliminated.

Devices affecting the properties of the radiated light may be configured in the zone of the coupling between the fiber optics hookup cable 15 and the light source 19 or of the coupling 16 between the distal segment 20 and the proximal segment 21 of the fiber optics bundle. These devices may control the emitted light's color, for instance being interference filters. This feature would especially apply to the pre-surgery stage wherein the operating room already has been darkened while many manual, functional endoscope steps are still required at this endoscope, namely by using red light, to make handling easier and simultaneously to substantially eliminate glare, while using white light during the intra-surgery stage to optimally illuminate the field of view. Again these devices may be designed to control light brightness, for instance being iris diaphragms, or to regularly interrupt the light flux, for instance being a chopper disk to generate a blinking light. These devices may be designed in a manner to be quickly turned On and OFF, for instance by means of a switch at the endoscope 10, illustratively, to make available full and undegraded light brightness at the beginning of surgery.

1. An endoscope (10) comprising an image transmitting system, a fiber optics bundle (20, 21), said bundle having a distal end segment, a distal end, and a proximal end and being mounted in the endoscope at least by the distal end segment (20) as to illuminate a field of view, said bundle, which radiates light from the distal end thereof to illuminate the field of view, is illuminated at the proximal end (18) by a light source (19), wherein said endoscope further comprises a light tapping system (22, 23, 26, 27, 28, 29) which taps light from the fiber optics bundle (20, 21) and guides the tapped light to a window (25, 30, 31, 32, 33) in an external wall of the endoscope (10).

2. The endoscope as claimed in claim 1, further comprising a hookup cable (15) that is fitted with a coupling (16) and can be disconnected from the endoscope (11), said hookup cable receiving a proximal end segment (21) of the fiber optics (20, 21), and wherein the window (25) is mounted in an external wall of the coupling (16).

3. The endoscope as claimed in claim 1, wherein an optic fiber (26, 27, 28, 29) branches off the fiber optics bundle (20, 21) and is guided to the window (30, 31, 32, 33).

4. The endoscope as claimed in claim 1, wherein the window (32, 33) is configured in one of a zone of a functional element (14, 13) and a zone of labeling of the endoscope (10).

5. The endoscope as claimed in claim 1, wherein the window is configured to illuminate at least one of a functional element (13) and a labeling of the endoscope (10).

6. The endoscope as claimed in claim 1, wherein several light tapping devices and windows are configured in the external wall of the endoscope (10) in a manner to subtend a pattern.
